SEQUENCE LISTING (28 sequences)

SEQ ID NO: 1 - DNA, 1653 bp, Artificial sequence
  Synthetic construct
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 2 - DNA, 324 bp, Artificial sequence
  Synthetic construct
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatt

SEQ ID NO: 3 - DNA, 315 bp, Artificial sequence
  Synthetic construct
ttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagg

SEQ ID NO: 4 - DNA, 313 bp, Artificial sequence
  Synthetic construct
atctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatac

SEQ ID NO: 5 - DNA, 327 bp, Artificial sequence
  Synthetic construct
gcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaag

SEQ ID NO: 6 - DNA, 320 bp, Artificial sequence
  Synthetic construct
ttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaa

SEQ ID NO: 7 - DNA, 364 bp, Artificial sequence
  Synthetic construct
taatacgactcactatagggtacatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggaggtaccctatagtgagtcgtatta

SEQ ID NO: 8 - DNA, 141 bp, Artificial sequence
  Synthetic construct
taatacgactcactatagggacaccccaacatcttcgacgcgggcgtggcaggtcttcccgacgatgacgccggtgaacttcccgccgccgttgttgttttggagcacggaaagacgatgaccctatagtgagtcgtatta

SEQ ID NO: 9 - DNA, 140 bp, Artificial sequence
  Synthetic construct
taatacgactcactatagggcactgataatgaattcctctggatctactgggttacctaagggtgtggcccttccgcatagaactgcctgcgtcagattctcgcatgccagagatcctatccctatagtgagtcgtatta

SEQ ID NO: 10 - DNA, 140 bp, Artificial sequence
  Synthetic construct
taatacgactcactatagggacacgaaattgcttctgggggcgcacctctttcgaaagaagtcggggaagcggttgcaaaacgcttccatcttccagggatacgacaaggatatgggctcccctatagtgagtcgtatta

SEQ ID NO: 11 - DNA, 140 bp, Artificial sequence
  Synthetic construct
taatacgactcactatagggaaaacataaagaaaggcccggcgccattctatcctctagaggatggaaccgctggagagcaactgcataaggctatgaagagatacgccctggttcctggccctatagtgagtcgtatta

SEQ ID NO: 12 - DNA, 140 bp, Artificial sequence
  Synthetic construct
taatacgactcactatagggaccagagtcctttgatcgtgacaaaacaattgcactgataatgaattcctctggatctactgggttacctaagggtgtggcccttccgcatagaactgccccctatagtgagtcgtatta

SEQ ID NO: 13 - DNA, 140 bp, Artificial sequence
  Synthetic construct
taatacgactcactatagggagaagtcggggaagcggttgcaaaacgcttccatcttccagggatacgacaaggatatgggctcactgagactacatcagctattctgattacacccgagccctatagtgagtcgtatta

SEQ ID NO: 14 - DNA, 141 bp, Artificial sequence
  Synthetic construct
taatacgactcactatagggcctaccgtagtgtttgtttccaaaaaggggttgcaaaaaattttgaacgtgcaaaaaaaattaccaataatccagaaaattattatcatggattctaaaacccctatagtgagtcgtatta

SEQ ID NO: 15 - DNA, 140 bp, Artificial sequence
  Synthetic construct
taatacgactcactatagggccatcacggttttggaatgtttactacactcggatatttgatatgtggatttcgagtcgtcttaatgtatagatttgaagaagagctgtttttacgatccccctatagtgagtcgtatta

SEQ ID NO: 16 - DNA, 140 bp, Artificial sequence
  Synthetic construct
taatacgactcactatagggaaattcaaagtgcgttgctagtaccaaccctattttcattcttcgccaaaagcactctgattgacaaatacgatttatctaatttacacgaaattgcttcccctatagtgagtcgtatta

SEQ ID NO: 17 - DNA, 140 bp, Artificial sequence
  Synthetic construct
taatacgactcactatagggccgttcggttggcagaagctatgaaacgatatgggctgaatacaaatcacagaatcgtcgtatgcagtgaaaactctcttcaattctttatgccggtgttccctatagtgagtcgtatta

SEQ ID NO: 18 - DNA, 2859 bp, Zea mays
  misc_feature (403)..(403): n is a, c, g, or t
gtaaaccggattattgtgcagccaagcacaaggaggatccatcatgacgctcagtatgaggatgcaggatgcgagatttcagaagacctgtcagaatgcggccttatcataggcatcaaacaacccaagctgcagatgattctttcagatagagcgtacgctttcttttcacacacacacaaagcccaaaaagagaatatgccactgttagacaagatccttgaagaaagggtgtccttgtttgattatgagctaattgttggagatgatgggaaaagatcactagcatttgggaaatttgctggtagagctggactgatagatttcttacatggtctcggacagcgatatttgagccttggatactcgactccatttctctctctgggacaatctcatatgntatccttcgctcgctgcagccaaggctgcagtcattgtcgttgcagaagagatagcaacatttggacttccatccggaatttgtccgatagtgtttgtgttcactggagttggaaacgtctctcagggtgcgcaggagatattcaagttattgccccatacctttgttgatgctgagaagcttcccgaaatttttcaggccaggaatctgtctaagcaatctcagtcgaccaagagagtatttcaactttatggttgtgttgtgacctctagagacatggtttctcacaaggatcccaccagacaatttgacaaaggtgactattatgctcatccagaacactacacccctgtttttcatgaaagaattgctccatatgcatctgtcatcgtaaactgtatgtattgggagaagaggtttccaccattactaaatatggatcagttacagcaattgatggagactggttgtcctttagtcggcgtttgtgacataacttgtgatattggaggttccattgaatttatcaacaagagtacatcaatagagaggcctttctttcggtatgatccttctaagaattcataccatgatgatatggaaggtgccggagtggtctgcttggctgttgacattctccctacagaattctctaaagaggcctcccaacattttggaaacatactatctagacttgttgctagtttggcctcagtgaagcaaccggcagaacttccttcctacttgagaagagcttgcattgcacatgctggcagattaactcctttgtatgaatatatccctaggatgagaaatactatgatagatttggcacccgcaaaaacaaatccattgcctgacaagaagtatagcaccctggtatctctcagtgggcacctatttgataagttccttataaatgaagctttggacatcattgagacagctggaggttcatttcacttggttagatgtgaagttggacaaagcacggatgatatgtcatactcagagcttgaagtaggagcagatgatactgccacattggataaaattattgattccttgacttctttagctaatgaacatggtggagatcacgatgccgggcaagaaattgaattagctctgaagataggaaaagtcaatgagtatgaaactgacgtcacaattgataaaggagggccaaagattttaattcttggagctggaagagtctgtcggccagctgctgagtttctggcatcttacccagacatatgtacctatggtgttgatgaccatgatgcagatcaaattcatgttatcgtggcatctttgtatcaaaaagatgcagaagagacagttgatggtattgaaaatacaactgctacccagcttgatgttgctgatattggaagcctttcagatcttgtttctcaggttgaggttgtaattagcttgctgcctgctagttttcatgctgccattgcaggagtatgcatagagttgaagaagcacatggtaacggcaagctatgttgatgaatccatgtcaaacttgagccaagctgccaaagatgcaggtgtaactatactttgtgaaatgggcctagatcctggcatagatcacttgatgtcaatgaagatgattgatgaagctcatgcacgaaagggaaaaataaaggcatttacatcttactgtggtggattgccatctccagctgcagcaaacaatccgcttgcctataaattcagttggaacccagctggtgcactccggtcagggaaaaatcctgcagtctacaaatttcttggtgagacgatccatgtagatggtcataacttgtatgaatcagcaaagaggctcagactacgagagcttccagcttttgctctggaacacttgccaaatcggaattccttgatatatggtgacctttatggtatctccaaagaagcatccaccatatatagggctactcttcgttacgaaggttttagtgagattatggtaaccctttccaaaactgggttctttgatgctgcaaatcatccactgctgcaagatactagtcgtccaacatataagggtttccttgatgaactactgaataatatctccacaattaacacggacttagatattgaagcctctggtggatacgatgatgacctgattgccagactgttgaagctcgggtgttgcaaaaataaggaaatagctgttaagacagtcaaaaccatcaagttcttgggactacatgaagagactcaaatacctaagggttgttcgagcccatttgatgtgatttgccagcgaatggaacagaggatggcctatggccacaatgagcaagacatggtactgctccaccacgaagtcgaggtggaatacccggacgggcaacccgccgaaaagcaccaagcgacgctactggagttcgggaaggttgaaaatggcaggtccaccactgc

SEQ ID NO: 19 - DNA, 555 bp, Artificial sequence
  Synthetic construct
aggctgcagtcattgtcgttgcagaagagatagcaacatttggacttccatccggaatttgtccgatagtgtttgtgttcactggagttggaaacgtctctcagggtgcgcaggagatattcaagttattgccccatacctttgttgatgctgagaagcttcccgaaatttttcaggccaggaatctgtctaagcaatctcagtcgaccaagagagtatttcaactttatggttgtgttgtgacctctagagacatggtttctcacaaggatcccaccagacaatttgacaaaggtgactattatgctcatccagaacactacacccctgtttttcatgaaagaattgctccatatgcatctgtcatcgtaaactgtatgtattgggagaagaggtttccaccattactaaatatggatcagttacagcaattgatggagactggttgtcctttagtcggcgtttgtgacataacttgtgatattggaggttccattgaatttatcaacaagagtacatcaatagagaggcctttctttcggtatgatccttcta

SEQ ID NO: 20 - DNA, 442 bp, Artificial sequence
  Synthetic construct
gcttggctgttgacattctccctacagaattctctaaagaggcctcccaacattttggaaacatactatctagacttgttgctagtttggcctcagtgaagcaaccggcagaacttccttcctacttgagaagagcttgcattgcacatgctggcagattaactcctttgtatgaatatatccctaggatgagaaatactatgatagatttggcacccgcaaaaacaaatccattgcctgacaagaagtatagcaccctggtatctctcagtgggcacctatttgataagttccttataaatgaagctttggacatcattgagacagctggaggttcatttcacttggttagatgtgaagttggacaaagcacggatgatatgtcatactcagagcttgaagtaggagcagatgatactgccacattggataaaattattga

SEQ ID NO: 21 - DNA, 98 bp, Artificial sequence
  Synthetic construct
tgaacatggtggagatcacgatgccgggcaagaaattgaattagctctgaagataggaaaagtcaatgagtatgaaactgacgtcacaattgataaag

SEQ ID NO: 22 - DNA, 1119 bp, Artificial sequence
  Synthetic construct
ttggagctggaagagtctgtcggccagctgctgagtttctggcatcttacccagacatatgtacctatggtgttgatgaccatgatgcagatcaaattcatgttatcgtggcatctttgtatcaaaaagatgcagaagagacagttgatggtattgaaaatacaactgctacccagcttgatgttgctgatattggaagcctttcagatcttgtttctcaggttgaggttgtaattagcttgctgcctgctagttttcatgctgccattgcaggagtatgcatagagttgaagaagcacatggtaacggcaagctatgttgatgaatccatgtcaaacttgagccaagctgccaaagatgcaggtgtaactatactttgtgaaatgggcctagatcctggcatagatcacttgatgtcaatgaagatgattgatgaagctcatgcacgaaagggaaaaataaaggcatttacatcttactgtggtggattgccatctccagctgcagcaaacaatccgcttgcctataaattcagttggaacccagctggtgcactccggtcagggaaaaatcctgcagtctacaaatttcttggtgagacgatccatgtagatggtcataacttgtatgaatcagcaaagaggctcagactacgagagcttccagcttttgctctggaacacttgccaaatcggaattccttgatatatggtgacctttatggtatctccaaagaagcatccaccatatatagggctactcttcgttacgaaggttttagtgagattatggtaaccctttccaaaactgggttctttgatgctgcaaatcatccactgctgcaagatactagtcgtccaacatataagggtttccttgatgaactactgaataatatctccacaattaacacggacttagatattgaagcctctggtggatacgatgatgacctgattgccagactgttgaagctcgggtgttgcaaaaataaggaaatagctgttaagacagtcaaaaccatcaagttcttgggactacatgaagagactcaaatacctaagggttgttcgagcccatttgatgtgatttgccagcgaatggaacagaggatggcct

SEQ ID NO: 23 - DNA, 115 bp, Artificial sequence
  Synthetic construct
tggtactgctccaccacgaagtcgaggtggaatacccggacgggcaacccgccgaaaagcaccaagcgacgctactggagttcgggaaggttgaaaatggcaggtccaccactgc

SEQ ID NO: 24 - DNA, 1860 bp, Diabrotica virgifera virgifera
gtcgacccacgcgtccgaatttgatggtgatcgtaccaaatatactatgttgcaagtatggcctgtacgtcaagcaaggccagtcagtgaaaaattacctgccaaccatcctctgcttacaggacagcgtgtacttgatgctcttttcccatgtgtacagggtggtactactgccattcccggagctttcggttgtggaaaaactgtaatttcacaatctctttccaaatattccaactctgatgtcattatctacgtcggttgcggagaaagaggtaacgaaatgtctgaagtattgagagatttccctgaattgactgttgaaattgacgggcacactgaatctattatgaaacgtaccgcattggtcgccaacacatctaacatgcctgtagctgctcgtgaagcttctatctatactggtattactctttctgaatacttccgtgatatgggttacaacgtatctatgatggctgactcgacatcacgttgggccgaagctttgagagaaatttcaggtcgtttggctgaaatgcctgccgattccggttatccggcttacttaggtgcccgtttggcttccttctacgaacgtgctggtcgcgttaaatgtttaggtaatccagacagagaaggatccgtttcaattgtaggagccgtatcacctcctggtggtgatttctcagatcctgttaccactgctactcttggtattgtacaggtgttctggggtttggacaagaaacttgcccaacgtaagcacttcccttcagtagactggcttggatcatattccaaatatttaagagcattggacgacttttatgacaaaaacttccaagagtttattcctcttagaaccaaagttaaggaaattcttcaggaagaagatgatctagccgaaattgtgcagctggtaggtaaagcatctctggcagaaacggacaaaatcaccttggaaattgccaggcttcttaaagaagatttcttgcaacaaaactcatactcttcttatgacagattctgtccattctataaaactgtcggtatgttgagaaacatgatcggtttgtacgacatggcgagacacgctgtagaatcaaccgcacaatcagaaaataagatcacttggaacgtaataagagattcaatgagtggaattttatatcaacttagcagtatgaaatttaaggatcccgtaaaagatggtgaagctaaaatcaaggcagattttgatcaattatatgaagatattcagcaggccttcagaaacttagaagattaaatctttttaaggaaattttcctattttgttcatcagtgtaagtttaaaaatatagcgatatttatcaaaaagaataataaggcctctatccctcacttctgtgaatattaatatggccgtactaaagatagtaactaaagataggttttctcttttttgatattatcctgtacaaaataaattatgtaaattgttgaatatgtgtatagtttttttgggtgagggtacagtgcttattaaatactttttaaacatttttcccgccattccaattactattaagttttttcgttttaatacttttttaaatatacaggtgcttaatatcgtttatattttcagtattacttggttttcttcatgtaaattgttttaaatttttcttttacccttttaatcttgtatattacattacccaattaaagttaattgtacagattaagataaacgagtatcttataacatctattagattgttagaatcaataaatgtagtgtaattgttctgttttgaacaaataaatgcatcaaaaaaaaaaaaaaaaaaaaaaaaaaggaaaaaaaaaaaaaaa

SEQ ID NO: 25 - DNA, 101 bp, Artificial sequence
  Synthetic construct
ggacaagaaacttgcccaacgtaagcacttcccttcagtagactggcttggatcatattccaaatatttaagagcattggacgacttttatgacaaaaact

SEQ ID NO: 26 - DNA, 138 bp, Artificial sequence
  Synthetic construct
ttattcctcttagaaccaaagttaaggaaattcttcaggaagaagatgatctagccgaaattgtgcagctggtaggtaaagcatctctggcagaaacggacaaaatcaccttggaaattgccaggcttcttaaagaag

SEQ ID NO: 27 - DNA, 170 bp, Artificial sequence
  Synthetic construct
acaaaactcatactcttcttatgacagattctgtccattctataaaactgtcggtatgttgagaaacatgatcggtttgtacgacatggcgagacacgctgtagaatcaaccgcacaatcagaaaataagatcacttggaacgtaataagagattcaatgagtggaattt

SEQ ID NO: 28 - DNA, 409 bp, Artificial sequence
  Synthetic construct
ggacaagaaacttgcccaacgtaagcacttcccttcagtagactggcttggatcatattccaaatatttaagagcattggacgacttttatgacaaaaactttattcctcttagaaccaaagttaaggaaattcttcaggaagaagatgatctagccgaaattgtgcagctggtaggtaaagcatctctggcagaaacggacaaaatcaccttggaaattgccaggcttcttaaagaagacaaaactcatactcttcttatgacagattctgtccattctataaaactgtcggtatgttgagaaacatgatcggtttgtacgacatggcgagacacgctgtagaatcaaccgcacaatcagaaaataagatcacttggaacgtaataagagattcaatgagtggaattt

(19) United States
(12) Patent Application Publication
(10) Pub. No.: US 2009/0117539 A1
(43) Pub. Date: May 7, 2009

(54) DNA sequences for gene suppression

(76) Inventors: Larry Gilbertson, Chesterfield, MO (US); Scott C. Johnson, Wildwood, MO (US); David K. Kovalic, Clayton, MO (US); Tracey L. Reynolds, Ballwin, MO (US); James F. Rice, JR., St. Charles, MO (US); James K. Roberts, Chesterfield, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/399,000

(22) Filed: Apr. 5, 2006

Related U.S. Application Data
(60) Provisional application No. 60/670,751, filed on Apr. 12, 2005.

(51) Int. Cl. C12Q 1/68; C12N 5/06

(52) U.S. Cl. 435/6; 435/325

(57) ABSTRACT

The present invention provides methods of improving DNA sequences for gene suppression mediated by double-stranded RNA, and constructs and transgenic organisms containing such improved DNA sequences.

### CROSS-REFERENCE TO RELATED APPLICATIONS AND INCORPORATION OF SEQUENCE LISTINGS

This application claims the benefit of priority of U.S. Provisional Patent Application 60/670,751, which was filed on 12 Apr. 2005, and is incorporated by reference in its entirety herein. The sequence listings contained in the file “38-21(53880)A.rpt” (file size of 19 KB in operating system MS-Windows, recorded on 12 Apr. 2005, and filed with U.S. Provisional Patent Application 60/670,751 on 12 Apr. 2005) is incorporated by reference in its entirety herein. The sequence listing contained in the file named “38-21(53880)B.rpt”, which is 20 kilobytes (measured in operating system MS-Windows) and located in computer readable form on a compact disk created on 4 Apr. 2006, is filed herewith and incorporated herein by reference.

### FIELD OF THE INVENTION

The present invention relates generally to the field of molecular biology, and more specifically discloses methods of improving DNA sequences for gene suppression mediated by double-stranded RNA, and constructs and transgenic organisms containing such improved DNA sequences.

### BACKGROUND OF THE INVENTION

Suppressing or silencing of genes in eukaryotes such as plants can occur at the transcriptional level or at the post-transcriptional level. Certain of these gene-suppressing mechanisms are associated with nucleic acid homology at the DNA or RNA level. See, for example, Matzke et al. (2001) Curr. Opin. Gen. Dev., 11:221-227 (2001), which is incorporated by reference in its entirety herein. Post-transcriptional gene silencing (PTGS) has emerged as the method of choice for silencing genes in plants. A number of versions of PTGS can be used, including anti-sense silencing, sense silencing (also known as co-suppression), and RNA interference (RNAi). All of these three gene suppression approaches are believed to operate through similar RNA processing pathways involving a double-stranded RNA (dsRNA) intermediate. Double-stranded RNA-mediated mechanisms are reviewed, for example, in Meister & Tuschl (2004) Nature, 431:343-349, which is incorporated by reference herein. In general, gene silencing can be achieved by the introduction of exogenous nucleic acids that generate a dsRNA which includes sequences that are identical, or nearly identical, to sequences of a specific target gene that is intended to be silenced. Typically, the target gene is an endogenous gene within the organism into which the exogenous nucleic acid is introduced, but the target gene can also be in any organism that may come into physical contact with the exogenous nucleic acid or with the dsRNA generated by the exogenous nucleic acid. For example, a corn rootworm gene could be the target gene when corn rootworm larvae consume plant tissues containing dsRNA corresponding to the corn rootworm gene.

The expression cassettes used in these three gene suppression approaches differ. In the case of antisense and sense silencing, a single-stranded transcript is converted to dsRNA through the action of RNA-dependent RNA polymerases (RdRP). In the case of RNAi, the RNAi gene cassette contains sequences (such as inverted repeats) within the transcribed region of the cassette, such that the initial transcript is at least partly self-complementary and can form dsRNA by self-annealing. For example, one approach for RNAi gene silencing in a plant is to make a transgenic plant that has an expression cassette containing within its transcribed region inverted repeats of a DNA sequence that is at least 19 base pairs long and that has substantial identity to the sequence of a gene that is the target for silencing.

In all these approaches, once dsRNA is formed, it is processed by RNase III enzymes called Dicer (or by Dicer-like proteins) into small double-stranded RNAs known as short interfering RNAs (siRNAs). The size of siRNAs is believed to range from about 20 to about 25 base pairs, but common classes of siRNAs include those containing 21 base pairs or 24 base pairs. See, for example, Hamilton et al. (2002) EMBO J., 21:4671-4679, which is incorporated by reference herein.

Different Dicer enzymes may produce a different size or class of siRNAs, which can range in size from 19 to 25 base pairs. For example, mammals are believed to contain a single Dicer gene, whereas plants are believed to contain multiple Dicer genes and to have two major size classes of siRNAs: 21 base pairs and 24 base pairs. With at least the exceptions of Dicer processing of microRNA (miRNA) precursors, and a recent example with a novel class of siRNAs (Vazquez, et al. (2004) Mol. Cell, 16:69-79, which is incorporated by reference herein), it is not known if there is any pattern to how Dicer cleaves dsRNA. Thus, for example, taking into consideration only the 21 base pair class of siRNAs, a 500 base pair dsRNA can theoretically produce 480 different siRNAs. There is no evidence yet that Dicer does not produce all theoretical siRNAs at equal frequencies. After double stranded siRNAs are generated by Dicer, one RNA strand is incorporated into the RNA-induced silencing complex (RISC). The strand that is selected by Dicer is believed to depend on certain thermodynamic properties of the double-stranded siRNA molecule, such as those described by Schwarz et al. (2003) Cell, 115:199-208, and Khvorova et al. (2003) Cell, 115:209-216, which are incorporated by reference in their entirety herein.

Apart from the effectiveness of a given DNA sequence to silence a gene by a dsRNA-mediated mechanism, considerations in the choice of the DNA sequence may include the potential of a DNA sequence to encode undesirable polypeptides (for example, polypeptides associated with protein-mediated allergenicity or toxicity), or the potential of a DNA sequence to produce functional siRNAs capable of interacting with and suppressing non-target genes (for example, genes that are not intended to be silenced, either in transgenic organisms or in organisms that may come into contact with siRNAs derived from the DNA sequence).

It would thus be useful to improve the efficiency and utility of gene silencing by selecting DNA sequences to be used in dsRNA gene suppression that are enhanced or improved over sequences that are randomly selected. Improvements to the selected DNA sequences can be any or a combination of an improvement in efficiency in dsRNA-mediated gene suppression, or a decreased potential to produce undesirable polypeptides, or a decreased potential to suppress non-target genes. The present invention provides novel methods of providing such improved DNA sequences useful in gene suppression or gene silencing mediated by double-stranded RNA (dsRNA). The present invention further provides transgenic eukaryotes (including, but not limited to, plants) whose genome includes at least one DNA sequence for enhanced dsRNA-mediated gene silencing provided by a method of the invention.

### SUMMARY OF THE INVENTION

The present invention discloses novel methods of providing improved DNA sequences useful, for example, for gene suppression or gene silencing mediated by double-stranded RNA (dsRNA).

In one aspect of this invention, the method includes the steps of (a) selecting from a target gene an initial DNA sequence including more than 21 nucleotides; (b) identifying at least one shorter DNA sequence derived from regions of the initial DNA sequence consisting of regions predicted to be more effective at dsRNA-mediated gene silencing, regions predicted to be more highly specific to the target gene; and regions predicted to not generate undesirable polypeptides; and (c) selecting a DNA sequence for dsRNA-mediated gene silencing that comprises the at least one shorter DNA sequence.

The invention also provides transgenic eukaryotes whose genome includes at least one DNA sequence for enhanced dsRNA-mediated gene silencing provided by the method of the present invention.

Other specific embodiments of the invention are disclosed in the following detailed description.

### BRIEF DESCRIPTION OF THE DRAWINGS

FIG. 1 depicts results of applying a computational algorithm for predicting gene silencing ability of a given sequence, as described in detail in Example 1. FIG. 1A depicts the moving average Reynolds score averaged for segments of 50 contiguous nucleotides over the length of the initial sequence (firefly luciferase coding region, SEQ ID NO. 1). FIG. 1B depicts the moving average Reynolds score averaged for segments of 100 contiguous nucleotides over the length of the initial sequence (SEQ ID NO. 1). FIG. 1C schematically depicts larger segments (SEQ ID NO. 2, SEQ ID NO. 3, SEQ ID NO. 4, SEQ ID NO. 5, and SEQ ID NO. 6), each of between about 300 and about 330 nucleotides, which together span the full length of SEQ ID NO. 1.

FIG. 2 depicts the moving average Reynolds score averaged for the five larger segments (SEQ ID NO. 2, SEQ ID NO. 3, SEQ ID NO. 4, SEQ ID NO. 5, and SEQ ID NO. 6), spanning the full length of SEQ ID NO. 1.

FIG. 3 depicts results of firefly luc suppression experiments in a maize protoplast model as described in Example 1. Results obtained for experiments using either 0.5 micrograms or 1 microgram of double-stranded RNA during the co-transformation are shown. The relative level of suppression of the target gene, firefly luciferase, is given as the logarithm of the ratio of firefly luciferase emission to Renilla luciferase emission, “log(Fluc/Rluc)”. The observed relative abilities of the segments to silence luciferase correlated well with the predicted siRNA efficiency ranking based on the average Reynolds scores of the segments.

FIG. 4 depicts results of firefly luc suppression experiments in a maize protoplast model as described in Example 1. Results obtained for experiments using 0.5 micrograms of double-stranded RNA during the co-transformation are shown. The relative level of suppression of the target gene, firefly luciferase, is given as the logarithm of the ratio of firefly luciferase emission to uidA (GUS) expression, “log(Fluc/GUS)”. The degree of firefly luc suppression matched the trend in the Reynolds scores calculated for these relatively large fragments (all substantially larger than 21 to 23 nucleotides). Segments High 1 through High 4 (SEQ ID NO. 14 through SEQ ID NO. 17), which had the highest average Reynolds scores, were consistently the most effective at suppressing luc expression, followed by the two Middle segments, Middle 1 (SEQ ID NO. 12) and Middle 2 (SEQ ID NO. 13), followed by Low 1 through Low 4 (SEQ ID NO. 8 through SEQ ID NO. 11), which were the least effective.

FIG. 5 depicts results of a bioinformatics (“Bfx”) analysis of a 2859 base pair initial DNA sequence (SEQ ID NO. 18), which was provided from plasmid pMON66619 and covered most of the 3183 base pair coding region of maize (Zea mays) lysine ketoglutarate reductase/saccharopine dehydrogenase (LKR/SDH), for regions predicted to be more highly specific to the target gene. This initial sequence was screened for matches to known vertebrate sequences. “Bfx-free zones” represent large contiguous areas where no 21/21 match to known vertebrate sequences was found.

FIG. 6 depicts results obtained in a corn rootworm assay for activity (shown as percent mortality) of DNA sequences. An improved DNA sequence for dsRNA-mediated gene silencing of the target gene, V-ATPase from Western corn rootworm (SEQ ID NO. 24), was obtained by selecting from an initial sequence three shorter DNA sequences consisting of regions predicted to be more highly specific to the target gene, and selecting a novel, chimeric DNA sequence (SEQ ID NO. 28) that includes the three shorter DNA sequences. Also shown are results for an untreated control (“UTC”, water) and a control sequence (“V-ATPase 6.0”, an approximately 290 base pair segment from the 3′ untranslated region of the V-ATPase gene).

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate aspects of the invention described by the plural of that term. The nomenclature used herein and the laboratory procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries. The inventors do not intend to be limited to a mechanism or mode of action. Reference thereto is provided for illustrative purposes only.

### Method of Providing DNA for Gene Silencing

The present invention provides a method to provide a DNA sequence for dsRNA-mediated gene silencing including the steps of (a) selecting from a target gene an initial DNA sequence including more than 21 nucleotides; (b) identifying at least one shorter DNA sequence derived from regions of the initial DNA sequence consisting of regions predicted to be more effective at dsRNA-mediated gene silencing, regions predicted to be more highly specific to the target gene; and regions predicted to not generate undesirable polypeptides; and (c) selecting a DNA sequence for dsRNA-mediated gene silencing that includes the at least one shorter DNA sequence.

The target gene may be any target gene of interest that can be suppressed by dsRNA-mediated gene silencing. Suitable target genes can include eukaryote genes such as, but not limited to, genes of plants, fungi, vertebrates, or invertebrates. Suitable target genes can include non-eukaryote genes, such as, but not limited to, genes of bacteria or of viruses. Target genes of particular interest include endogenous genes of plants including, but not limited to, agriculturally or commercially important plants, including monocots and dicots, especially crop plants, wood- or pulp-producing trees, vegetable plants, fruit plants, and ornamental plants (for example, monocots including turf and forage grasses and crops such as maize, wheat, oat, barley, rye, triticale, sorghum, millet, sugarcane, and rice, more preferably maize, wheat, and rice, and dicots including canola, cotton, potato, quinoa, amaranth, buckwheat, safflower, soybean, sugarbeet, and sunflower, more preferably soybean and cotton), as well as endogenous genes of plant pests and pathogens (e.g., invertebrates such as arthropods or nematodes, fungi, bacteria, and viruses). Genes from nematodes or arthropods may include genes for major sperm protein, alpha tubulin, V-ATPase, RNA polymerase II, chitin synthase, as well as other genes such as those disclosed in Table II of United States Patent Application Publication 2004/0098761 A1, which is incorporated by reference in its entirety herein. Additional examples of potential target genes from plant pests and pathogens, as well as methods for providing and assaying gene suppression sequences for such target genes, are disclosed in United States Patent Application Publication 2006/0021087 A1 to Baum et al., entitled “Compositions and Methods for Control of Insect Infestations in Plants”, filed on 8 Apr. 2005, which is incorporated by reference in its entirety herein. Thus, a target gene of interest need not be endogenous to the organism in which the dsRNA is expressed. For example, it has been demonstrated that dsRNA expressed in bacteria was capable of silencing a target gene expressed in nematodes that fed on the bacteria (see, for example, U.S. Pat. No. 6,506,559, and Timmons and Fire (1998) Nature, 395:854, which are incorporated by reference herein). Thus, the present invention envisions, for example, that a DNA sequence for dsRNA-mediated gene silencing obtained by the method of the invention may be transcribed in a plant and used to suppress a gene of a pathogen or pest that may infest the plant.

In a preferred embodiment of the invention, the initial DNA sequence includes more than 21 nucleotides. Generally speaking, there is no upper size limit to the initial DNA sequence. Thus, the initial DNA sequence can include, for example, more than 22, or more than 23, or more than 24 nucleotides. In some preferred embodiments, the initial DNA sequence can be substantially larger than 21 nucleotides, for example, larger than about 50, about 100, about 300, about 500, about 1000, about 3000, or about 5000 nucleotides or greater.

At least one shorter DNA sequence is derived from regions of the initial DNA sequence. This shorter DNA sequence can be derived from regions predicted to be more effective at dsRNA-mediated gene silencing, or from regions predicted to be more highly specific to the target gene, or from regions predicted to not generate undesirable polypeptides, or from a combination of any of these. The shorter DNA sequence preferably includes at least 19 nucleotides, and in some embodiments preferably includes at least 21, at least 22, at least 23, or at least 24 nucleotides. For example, where a functional siRNA is believed to require 21 nucleotides, the shorter DNA sequence preferably includes at least 21 nucleotides.

The regions predicted to be more effective at dsRNA-mediated gene silencing include regions predicted to have higher siRNA efficiency. Such predictions of higher siRNA efficiency may be made by any technique, including, but not limited to, computational methods such as algorithms designed to predict siRNA efficiency based on thermodynamic characteristics of a given dsRNA (or DNA) sequence. Current predictions generally consider sequences of about 19 to about 21 base pairs.

For example, at least eight criteria for predicting the efficiency at which a given siRNA would function have been identified by Reynolds et al. (see Reynolds et al. (2004) Nature Biotechnol., 22:326-330, which is incorporated by reference in its entirety herein). Based on the Reynolds criteria, a numerical “Reynolds score” can be assigned to a given theoretical siRNA sequence. The Reynolds criteria and scoring guidelines include:

1. 30%-52% G/C content (+1 if true)

2. At least 3 A/U bases at position 15-19 (+1 for each)

3. Absence of internal repeats (Reynolds predicts this based on predicting a melting temperature or Tₘ of less than or equal to 20 degrees, a substitute approach can be made by predicting a minimum free energy greater than −4) (+1 if true)

4. An A base at position 19 (+1 if true)

5. An A base at position 3 (+1 if true)

6. A U base at position 10 (+1 if true)

7. A base other than G or C at position 19 (−1 if false)

8. A base other than G at position 13 (−1 if false)

The Reynolds criteria are examples of methods for predicting the effectiveness of a given sequence in dsRNA-mediated gene silencing. Other “siRNA prediction” tools exist and are publicly available. Non-limiting examples of these include “siSearch—An siRNA design Program” (publicly available at sonnhammer.cgb.ki.se/siSearch/siSearch_{—}1.6.html); Yuan et al. (2004) Nucleic Acids Res., 32:130-134; Chalk et al. (2004) Biochem. Biophys. Res. Commun., 319:264-274; Sætrom and Snove (2004) Biochem. Biophys. Res. Commun., 321: 247-253; Cui et al. (2004) Comput. Methods Programs Biomed., 75:67-73; Henschel et al. (2004) Nucleic Acids Res., 32:W113-120 (Web Server issue); Patzel, V (2004) Curr. Opin. Drug Discov. Devel., 7:360-369; Boese et al. (2005) Methods Enzymol, 392:73-96; and Heale et al. (2005) Nucleic Acids Res., 33:30, all of which are incorporated by reference herein. These and other prediction techniques, including improved and future prediction techniques, may be used in the method of the invention.

These siRNA prediction tools are generally designed to assist researchers to select individual siRNAs to test in gene suppressing experiments. This is useful and relatively easy to apply when the experimental approach is to use a single, synthetic siRNA made up of two short (typically 21 nucleotides), complementary or substantially complementary RNA oligonucleotides annealed together. However, gene silencing mediated by dsRNA is commonly practiced by transgenically expressing dsRNA of greater than the size of a typical single siRNA of 21 base pairs. It has not been established if these siRNA prediction tools can be applied to experimental approaches in which a relatively large dsRNA (for example a dsRNA larger than a typical single siRNA of 21 base pairs) is delivered to the cell or is produced in vivo. It is not obvious that any of these siRNA prediction tools can be successfully applied to dsRNAs longer than the length of a single siRNA, because of the apparent randomness of Dicer activity. Where the size of the dsRNA is greater than 21 base pairs, multiple theoretical siRNAs can be produced. For example, Dicer can theoretically produce 80 different siRNAs from a 100 base pair dsRNA sequence.

The present invention provides a novel technique for screening regions of the initial DNA sequence for regions predicted to be more effective at dsRNA-mediated gene silencing. In this novel technique, an siRNA prediction tool is applied to dsRNAs longer than the length of a single siRNA (that is, generally longer than 21 nucleotides), and, in some embodiments, to dsRNAs substantially longer than the length of a single siRNA. In one non-limiting example, based on the Reynolds criteria, a given initial DNA sequence, such as a sequence of a gene targeted for suppression, can be screened. Each theoretical siRNA is scored by the eight criteria, and a final composite Reynolds score (that is, a composite predicted siRNA effectiveness) is assigned to each screened initial DNA sequence (or region of the initial DNA sequence larger than 21 nucleotides). The composite Reynolds score may be based at least partly on, for example, an average or median Reynolds score from all or a subset of all theoretical siRNAs. In another embodiment, a region predicted to be more effective at dsRNA-mediated gene silencing may include a region containing a relatively high abundance of individual theoretical siRNAs having a Reynolds score greater than a selected value (for example, a Reynolds score greater than about 5, about 6, about 7, or even higher).

The regions predicted to be more effective at dsRNA-mediated gene silencing also include regions predicted to be more highly specific to the target gene. Regions predicted to be more highly specific to the target gene include regions substantially non-identical to a non-target gene sequence. Non-target genes can include any gene not intended to be silenced or suppressed, either in the transgenic organisms expressing the dsRNA or in organisms that may come into contact with siRNAs produced by the dsRNA. A non-target gene sequence can include any sequence from any species (including, but not limited to, non-eukaryotes such as bacteria, and viruses; fungi; plants, including monocots and dicots, such as crop plants, ornamental plants, and non-domesticated or wild plants; invertebrates such as arthropods, annelids, nematodes, and molluscs; and vertebrates such as amphibians, fish, birds, domestic or wild mammals, and even humans).

In one embodiment of the invention, the target gene is a gene endogenous to a given species, such as a given plant (such as, but not limited to, agriculturally or commercially important plants, including monocots and dicots), and the non-target gene can be, for example, a gene of a non-target species, such as another plant species or a gene of a virus, fungus, bacterium, invertebrate, or vertebrate, even a human. One non-limiting example of this embodiment is where a region predicted to be more effective at dsRNA-mediated gene silencing in a single species (e.g., Western corn rootworm, Diabrotica virgifera virgifera LeConte) contains a region that includes sequence specific to a gene endogenous to that single species, and that does not include sequence from genes from related, even closely related, species (e.g., Northern corn rootworm, Diabrotica barberi Smith and Lawrence, or Southern corn rootworm, Diabrotica undecimpunctata).

In other embodiments (for example, where the target gene is intended to have applications across multiple species), it may be desirable for the regions predicted to be more highly specific to the target gene to be selected from regions that contain sequence common to multiple species in which the target gene is to be silenced. Thus, a DNA sequence for dsRNA-mediated gene silencing may be selected to be specific for one taxon (for example, specific to a genus, family, or even a larger taxon such as a phylum, e.g., arthropoda) but not for other taxa (for example, plants or vertebrates or mammals). In one non-limiting example of this embodiment, a DNA sequence for dsRNA-mediated gene silencing in corn rootworm may be selected to be specific to all members of the genus Diabrotica. In a further example of this embodiment, such a Diabrotica-targeted DNA sequence may be selected so as to not contain any sequence from beneficial coleopterans (for example, predatory coccinellid beetles, commonly known as ladybugs or ladybirds) or other beneficial insect species.

The required degree of specificity of a DNA sequence for dsRNA-mediated gene silencing depends on various factors, including the size of the smaller dsRNA fragments that are expected to be produced by the action of Dicer, and the relative importance of decreasing the dsRNA's potential to suppress non-target genes. For example, where the dsRNA fragments are expected to be 21 base pairs in size, one particularly preferred embodiment of regions substantially non-identical to a non-target gene sequence includes regions within which every contiguous fragment including at least 21 nucleotides matches fewer than 21 (e.g., fewer than 21, or fewer than 20, or fewer than 19, or fewer than 18, or fewer than 17) out of 21 contiguous nucleotides of a non-target gene sequence. In another embodiment, regions substantially non-identical to a non-target gene sequence includes regions within which every contiguous fragment including at least 19 nucleotides matches fewer than 19 (e.g., fewer than 19, or fewer than 18, or fewer than 17, or fewer than 16) out of 19 contiguous nucleotides of a non-target gene sequence.

The regions predicted to be more effective at dsRNA-mediated gene silencing can further include regions predicted to not generate undesirable polypeptides. For example, in many cases the transcribed region of a gene cassette that produces a double stranded RNA is not intended to be translated into a polypeptide, but may have the potential to be translated into one or more polypeptides, since, in most cases, the transcript would be expected to have a 5′ cap and a polyadenylated 3′ end. The present invention includes a novel technique for screening regions of the initial DNA sequence for regions predicted to be more effective at dsRNA-mediated gene silencing and consisting of regions predicted to not generate undesirable polypeptides. Sequences of the transcribed region of a gene cassette intended to produce a double stranded RNA can be screened, for example, for sequences that may encode known undesirable polypeptides or close homologues of these. Undesirable polypeptides include, but are not limited to, polypeptides homologous to known allergenic polypeptides and polypeptides homologous to known polypeptide toxins. Publicly available sequences encoding such undesirable potentially allergenic peptides are available, for example, the Food Allergy Research and Resource Program (FARRP) allergen database (available at allergenonline.com) or the Biotechnology Information for Food Safety Databases (available at www.iit.edu/˜sgendel/fa.htm) (see also, for example, Gendel (1998) Adv. Food Nutr. Res., 42:63-92, which is incorporated by reference herein). Undesirable sequences can also include, for example, those polypeptide sequences annotated as known toxins or as potential or known allergens and contained in publicly available databases such as GenBank, EMBL, SwissProt, and others, which are searchable by the Entrez system (www.ncbi.nih.gov/Entrez). Non-limiting examples of undesirable, potentially allergenic peptide sequences include glycinin from soybean, oleosin and agglutinin from peanut, glutenins from wheat, casein, lactalbumin, and lactoglobulin from bovine milk, and tropomysosin from various shellfish (allergenonline.com). Non-limiting examples of undesirable, potentially toxic peptides include tetanus toxin tetA from Clostridium tetani, diarrheal toxins from Staphylococcus aureus, and venoms such as conotoxins from Conus spp. and neurotoxins from arthropods and reptiles (www.ncbi.nih.gov/Entrez).

In one non-limiting example, Applicants have screened potential dsRNA sequences to eliminate those sequences encoding polypeptides with perfect homology to a known allergen or toxin over 8 contiguous amino acids, or with at least 35% identity over at least 80 amino acids; such screens can be performed on any and all possible reading frames in both directions, on potential open reading frames that begin with ATG, or on all possible reading frames, regardless of whether they start with an ATG or not.

In a non-limiting example, Applicants have routinely performed screens (referred to as “EAT/Tox” screens) on the transcribed portions of the gene cassettes that are intended to produce a double stranded RNA but are not intended to be translated into a polypeptide. These screens can be performed on any and all possible reading frames in both directions, and on potential open reading frames that begin with ATG, or on all possible reading frames, regardless of whether they start with an ATG or not. When a “hit” or match is made, that is, when a sequence that encodes a potential polypeptide with perfect homology to a known allergen or toxin over 8 contiguous amino acids (or at least about 35% identity over at least about 80 amino acids), is identified, the DNA sequences corresponding to the hit can be avoided, eliminated, or modified when selecting sequences to be used for dsRNA gene silencing.

Avoiding, elimination of, or modification of, an undesired sequence may be achieved by any of a number of methods known to those skilled in the art. In some cases, the result may be novel sequences that are believed to not exist naturally. For example, avoiding certain sequences can be accomplished by joining together “clean” sequences into novel chimeric sequences that will produce a novel transcript, most preferably a novel transcript that will function in a normal dsRNA-mediated silencing pathway.

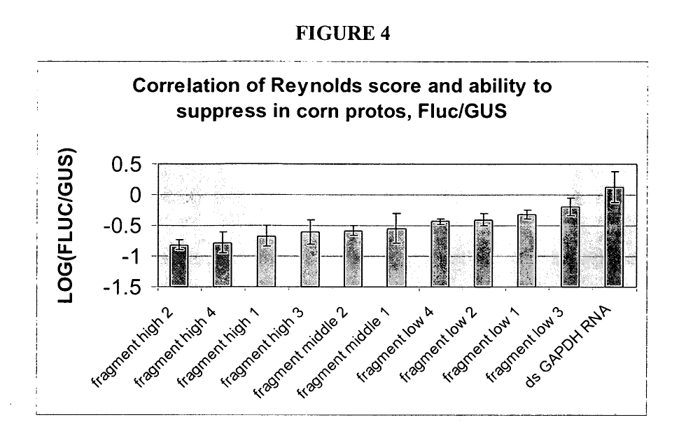

Applicants recognize that in some dsRNA-mediated gene silencing, it is possible for imperfectly matching dsRNA sequences to be effective at gene silencing. For example, it has been shown that mismatches near the center of a miRNA complementary site has stronger effects on the miRNA's gene silencing than do more distally located mismatches. See, for example, FIG. 4 in Mallory et al. (2004) EMBO J., 23:3356-3364, which is incorporated by reference herein. In another example, it has been reported that, both the position of a mismatched base pair and the identity of the nucleotides forming the mismatch influence the ability of a given siRNA to silence a target gene, and that adenine-cytosine mismatches, in addition to the G:U wobble base pair, were well tolerated (see Du et al. (2005) Nucleic Acids Res., 33:1671-1677, which is incorporated by reference herein). Thus, a DNA sequence for dsRNA-mediated gene silencing need not always have 100% sequence identity with the intended target gene, but generally would preferably have substantial sequence identity with the intended target gene, such as about 95%, about 90%, about 85%, or about 80% sequence identity with the intended target gene. One skilled in the art would be capable of judging the importance given to screening for regions predicted to be more highly specific to the target gene or predicted to not generate undesirable polypeptides, relative to the importance given to other criteria, such as, but not limited to, the percent sequence identity with the intended target gene or the predicted gene silencing efficiency of a given sequence. For example, it may be desirable for a given DNA sequence for dsRNA-mediated gene silencing to be active across several species, and therefore one skilled in the art may determine that it is more important to include regions specific to the several species of interest, but less important to screen for regions predicted to have higher gene silencing efficiency or for regions predicted to generate undesirable polypeptides.

The DNA sequence for dsRNA-mediated gene silencing includes at least one shorter DNA sequence derived from regions of the initial DNA as described in this method, and therefore includes at least 19 nucleotides, and can include substantially more than 19 nucleotides. In one embodiment, the DNA sequence for dsRNA-mediated gene silencing thus includes at least one shorter DNA sequence derived from regions of the initial DNA can include at least about 50, about 100, about 300, about 500, about 1000, or about 3000 nucleotides or greater. The DNA sequence for dsRNA-mediated gene silencing can include multiple shorter DNA sequences derived from regions of the initial DNA. The single or multiple shorter DNA sequences can be selected using any or a combination of the selection methods described. Where multiple shorter DNA sequences are selected, they can be combined, sequentially or overlapping, in any order, into a single chimeric DNA sequence for dsRNA-mediated gene silencing.

### Transgenic Eukaryotes

The present invention further discloses and provides a transgenic eukaryote whose genome includes at least one DNA sequence for dsRNA-mediated gene silencing provided by the method described above under the heading “Method of Providing DNA for Gene Silencing”. Any suitable eukaryote may be made transgenic for such a DNA sequence. Suitable eukaryotes include, but are not limited to, plants (for example, monocots and dicots, including crop plants, ornamental plants, and non-domesticated or wild plants); domestic or wild mammals, birds, and fish; invertebrates such as arthropods and nematodes; yeasts and fungi. Of particular interest are plants of commercial or agricultural interest, such as crop plants, wood- or pulp-producing trees, vegetable plants, fruit plants, and ornamental plants, and pests, pathogens, or symbionts of such plants. Preferred dicot plants include, but are not limited to, canola, cotton, potato, quinoa, amaranth, buckwheat, safflower, soybean, sugarbeet, and sunflower, more preferably soybean, canola, and cotton. In a particularly preferred embodiment, the transgenic plant is a transgenic monocot plant, more preferably a transgenic monocot crop plant, such as, but not limited to, wheat, oat, barley, maize, rye, triticale, rice, ornamental and forage grasses, sorghum, millet, and sugarcane, more preferably maize, wheat, and rice. The transgene for which the eukaryote is transgenic can be any target gene of interest (including eukaryote genes or non-eukaryote genes). The transgene can target a gene endogenous to the transgenic eukaryote, or endogenous to a species other than the transgenic eukaryote, and can include multiple target genes.

### EXAMPLES

### Example 1

This example is a non-limiting example of a method to provide a DNA sequence for dsRNA-mediated gene silencing. More specifically, this example describes selection of an improved DNA useful in dsRNA-mediated gene silencing by (a) selecting from a target gene an initial DNA sequence including more than 21 nucleotides; (b) identifying at least one shorter DNA sequence derived from regions of the initial DNA sequence consisting of regions predicted to be more effective at dsRNA-mediated gene silencing; and (c) selecting a DNA sequence for dsRNA-mediated gene silencing that includes the at least one shorter DNA sequence.

The coding region of the target gene, firefly luciferase (luc) (SEQ ID NO. 1), was chosen as an initial DNA sequence including more than 21 nucleotides. A prediction tool was developed that applied the criteria of an siRNA prediction algorithm (described in detail by Reynolds et al. (2004) Nature Biotechnol., 22:326-330, which is incorporated by reference in its entirety herein) to any DNA sequence (including DNA sequences larger than 21 or 23 nucleotides). This prediction tool was applied to the initial DNA sequence. Other suitable prediction algorithms, including current prediction tools as well as new or improved prediction tools developed in the future, may be used in place of the prediction tool using the Reynolds algorithm.

The Reynolds score was calculated for all potential siRNAs (that is, all possible segments containing 21 contiguous nucleotides of the initial DNA sequence, SEQ ID NO. 1), and these scores were averaged for larger segments of the initial sequence. The higher the Reynolds score, the more efficient that particular siRNA was predicted to be. Individual Reynolds scores for siRNAs from the SEQ ID NO. 1 ranged from about −2 to about 10. The average Reynolds score over the entire length of SEQ ID NO. 1 was 4.32. Alternative approaches could include calculating a median Reynolds score, or determining the abundance of individual theoretical siRNAs having a Reynolds score greater than a selected value (for example, a Reynolds score greater than about 5, about 6, about 7, or even higher).

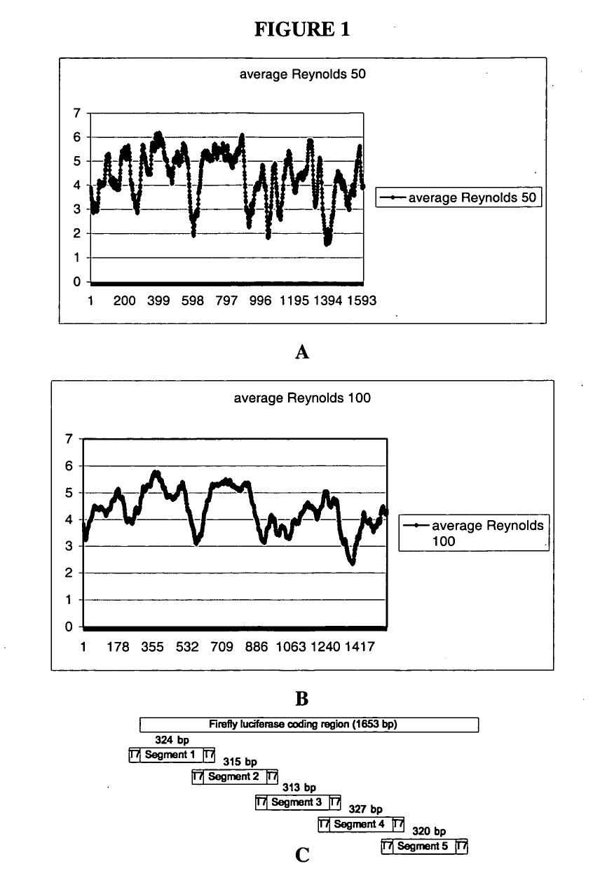

Sizes of dsRNA used for gene silencing are often longer than the size of a single siRNA (about 21 to about 23 nucleotides). For example, a commonly used size of dsRNA for silencing is about 100 base pairs. Thus, this computational approach to predicting regions more effective at dsRNA-mediated gene silencing was applied to segments of 50 base pairs or of 100 base pairs, over the length of the initial sequence. FIG. 1A depicts the moving average Reynolds score averaged for 50-mer segments (segments of 50 contiguous nucleotides) over the length of the initial sequence. The magnitude of the difference between the highest and lowest average Reynolds score is about 4.66. FIG. 1B depicts the moving average Reynolds score averaged for 100-mer segments (segments of 100 contiguous nucleotides) over the length of the initial sequence. The magnitude of the difference between the highest and lowest average Reynolds score is about 3.45.

In this example, the average Reynolds score for individual 21-mer potential siRNAs varied substantially along the length SEQ ID NO. 1 (FIGS. 1A and 1B). In the analysis of 100-mer segments, average Reynolds scores ranged from a low score of 2.33 for a 100 base pair segment beginning at nucleotide position 1380, to a score of 5.78 for a 100 base pair segment beginning at nucleotide position 367. Thus, in this example, a segment that corresponds to sequences that begin at nucleotide position 367 would be predicted to be more effective at dsRNA-mediated gene silencing than most other possible 100 base pair sequences. It is anticipated that such larger segments (greater than about 21 to about 23 base pairs) with the highest predicted ability to silence a target gene will not always be, in fact, the most effective or the most preferred segment; however, on average, segments with higher predicted silencing ability are expected to be more effective at dsRNA-mediated gene silencing than are randomly chosen segments of equal size. Thus, this type of analysis has utility in the selection of sequences to use in dsRNA-mediated gene silencing methods. Anti-sense and sense suppression methods are also believed to operate through dsRNA intermediates, and thus it is anticipated that this analysis will have further utility for selection of sequences for those methods.

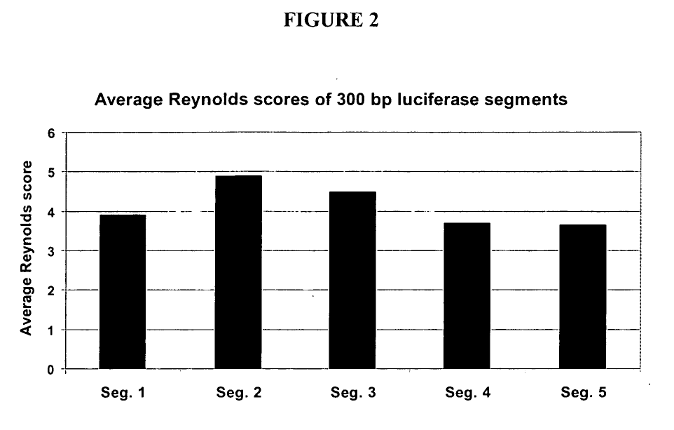

This computational approach to predicting regions more effective at dsRNA-mediated gene silencing was applied to even larger segments of greater than about 300 nucleotides. Table 1 lists five segments (SEQ ID NO. 2, SEQ ID NO. 3, SEQ ID NO. 4, SEQ ID NO. 5, and SEQ ID NO. 6), each of between about 300 and about 330 nucleotides, which together span most of the length of SEQ ID NO. 1. These larger segments are depicted schematically in FIG. 1C. The average Reynolds score for each of these segments ranged from about 3.66 (segment number 5, SEQ ID NO. 6) to about 4.91 (segment number 2, SEQ ID NO. 3), and is depicted graphically in FIG. 2. Based on their average Reynolds score, these five segments would be predicted to rank as 2>3>1>4>5 (by segment number) for their ability to silence firefly luc expression. The magnitude of the difference between the highest and lowest average Reynolds score is about 1.25.

TABLE 1

| | | nucleotide | | average |
|---|---|---|---|---|
| segment | | position in | size | Reynolds |
| number | SEQ ID NO. | luc gene | (bp) | score |
| (full length luc) | 1 | 1 to 1653 | 1653 | 4.32 |
| 1 | 2 | 1 to 324 | 324 | 3.92 |
| 2 | 3 | 305 to 619 | 315 | 4.91 |
| 3 | 4 | 600 to 912 | 313 | 4.51 |
| 4 | 5 | 893 to 1219 | 327 | 3.69 |
| 5 | 6 | 1200 to 1519 | 320 | 3.66 |

Luciferase suppression experiments were carried out with a transfection assay using a maize protoplast model system. Maize protoplasts were prepared as previously described by Sheen (1990) Plant Cell, 2:1027-1038, which is incorporated by reference herein. Polyethylene glycol (PEG)-mediated transformations (see, for example, Armstrong et al. (1990), Plant Cell Rep., 9:335-339, which is incorporated by reference herein) were performed in deep well (2 milliliters/well) 96-well plates. Separate vectors containing either firefly luciferase or Renilla luciferase were employed as reporters. The firefly luciferase reporter vector included a chimeric promoter including a chimeric promoter including an enhanced cauliflower mosaic virus (CaMV) 35S promoter linked to an enhancer element (an intron from heat shock protein 70 of Zea mays), the coding sequence of the firefly luciferase gene luc (SEQ ID NO. 1), and a 3′ untranslated region (3′ UTR) DNA from Agrobacterium tumefaciens nopaline synthase gene (3′NOS) which provides a polyadenylation (polyA) site. The Renilla luciferase reporter vector included the same chimeric promoter and 3′NOS UTR terminator. Generally, 1.3 micrograms firefly luciferase reporter vector DNA, 0.6 micrograms Renilla luciferase reporter vector DNA, and additional plasmid (pUC18) DNA were added to each well in order to maintain the total amount of RNA plus DNA constant at 12.5 micrograms per well. To each well was added 160 microliters (2×10⁶ protoplasts per milliliter) of maize protoplasts. Protoplasts were made transformation-competent by treatment with a solution containing 4 grams PEG 4000, 2 milliliters water, 3 milliliters 0.8 molar mannitol, and 1 milliliter Ca(NO₃)₂.

The five dsRNAs tested in the protoplast model were all about 365 base pairs in size and included a shorter DNA sequence derived from regions of the initial DNA sequence (i.e., segments 1-5) flanked on each side by the RNA polymerase T7 promoter sequence. The protoplasts were co-transformed with the double-stranded RNA segments, together with the reporter vectors for firefly luciferase and Renilla luciferase, into 4 separate volumes of maize protoplasts. The relative level of suppression of the target gene, firefly luciferase, was indicated by the intensity of firefly luciferase emission (“Fluc”) normalized to Renilla luciferase emission (Rluc). A 364 base pair dsRNA (SEQ ID NO. 7) including a 318 base pair segment that consisted of positions 1 through 318 from the gene for beta-glucuronidase (GUS) (uidA), flanked on each end by 23 base pairs of the RNA polymerase T7 promoter sequence, was used as a negative control dsRNA. The full-length luc gene (SEQ ID NO. 1) was used as a positive control dsRNA.

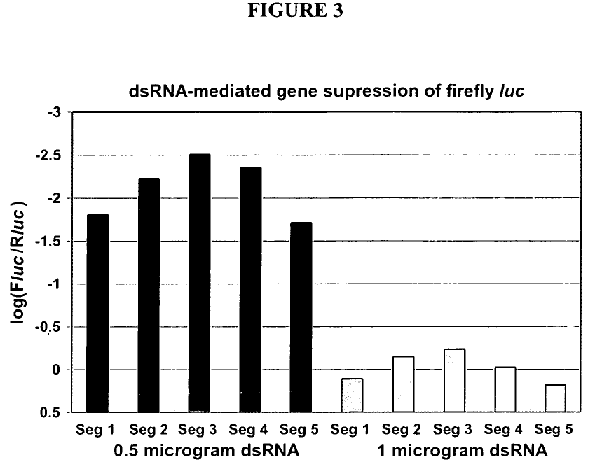

FIG. 3 depicts results of firefly luc suppression experiments using either 0.5 micrograms or 1 microgram of double-stranded RNA during the co-transformation. The relative level of suppression of the target gene, firefly luciferase, is given as the logarithm of the ratio of firefly luciferase emission to Renilla luciferase emission, “log(Fluc/Rluc)”. The degree of firefly luc suppression was similar to the trend in the Reynolds scores calculated for these relatively large fragments (all substantially larger than 21 to 23 nucleotides). Segment 5 (SEQ ID NO. 6), which had the lowest average Reynolds score, was consistently the dsRNA that was least effective at suppressing luc expression. Segment 3 (SEQ ID NO. 4), the most effective suppressor, had one of the highest average Reynolds scores. The observed relative abilities of the segments to silence luciferase correlated well with the predicted siRNA efficiency ranking based on the average Reynolds scores of the segments.

In another experiment, ten dsRNAs tested in the protoplast model were all 140 or 141 base pairs in size and included a shorter 100 or 101 base pair DNA sequence derived from regions of the initial DNA sequence (SEQ ID NO. 1) flanked on each side by the RNA polymerase T7 promoter sequence. The regions selected are listed in Table 2. Regions were selected based on their average Reynolds scores, using 100 base pairs segments sizes (FIG. 1B). Selecting sequences of 100 base pairs resulted in segments with relatively large difference in their average Reynolds score. The four lowest scoring regions were selected (Low 1 through Low 4, SEQ ID NO. 8 through SEQ ID NO. 11), the four highest scoring regions were selected (High 1 through High 4, SEQ ID NO. 14 through SEQ ID NO. 17), and two regions with scores that were close to the average score for the entire luciferase gene were selected (Middle 1, SEQ ID NO. 12, and Middle 2, SEQ ID NO. 13). As a negative control, a 500 base pair dsRNA corresponding to the GAPDH gene was used (Ambion Silencer® siRNA Cocktail Kit, catalog #1612). The protoplasts were co-transformed with the double-stranded RNA segments, together with the reporter vectors for firefly luciferase and the uidA (GUS) gene, into 4 separate volumes of maize protoplasts. The relative level of suppression of the target gene, firefly luciferase, was indicated by the intensity of firefly luciferase emission (“Fluc”) normalized to uidA expression.

TABLE 2

| | | nucleotide | |
|---|---|---|---|
| | | position in luc | average |
| segment name | SEQ ID NO. | gene | Reynolds score |
| Low 1 | 8 | 1380-1480 | 2.33 |
| Low 2 | 9 | 578-677 | 3.10 |
| Low 3 | 10 | 927-1026 | 3.12 |
| Low 4 | 11 | 14-113 | 3.23 |
| Middle 1 | 12 | 546-645 | 4.18 |
| Middle 2 | 13 | 963-1062 | 4.18 |
| High 1 | 14 | 367-467 | 5.78 |
| High 2 | 15 | 729-828 | 5.50 |
| High 3 | 16 | 842-941 | 5.38 |
| High 4 | 17 | 179-278 | 5.15 |

FIG. 4 depicts results of firefly luc suppression experiments using 0.5 micrograms of double-stranded RNA during the co-transformation. The relative level of suppression of the target gene, firefly luciferase, is given as the logarithm of the ratio of firefly luciferase emission to uidA (GUS) expression, “log(Fluc/GUS)”. The degree of firefly luc suppression matched the trend in the Reynolds scores calculated for these relatively large fragments (all substantially larger than 21 to 23 nucleotides). Segments High 1 through High 4 (SEQ ID NO. 14 through SEQ ID NO. 17), which had the highest average Reynolds scores, were consistently the most effective at suppressing luc expression, followed by the two Middle segments, Middle 1 (SEQ ID NO. 12) and Middle 2 (SEQ ID NO. 13), followed by Low 1 through Low 4 (SEQ ID NO. 8 through SEQ ID NO. 11), which were the least effective.

These results demonstrated that an improved DNA useful in dsRNA-mediated gene silencing was be obtained by selecting from a target gene an initial DNA sequence larger than 21 nucleotides (and larger than at least between about 300 and about 330 nucleotides), identifying at least one shorter DNA sequence derived from regions predicted to be more effective at dsRNA-mediated gene silencing (for example, by an computational algorithm), and selecting a DNA sequence for dsRNA-mediated gene silencing that includes the at least one shorter DNA sequence.

As these non-limiting examples demonstrate, an algorithm designed to predict the efficiency of individual siRNAs (of 21 base pairs) was successfully applied to longer dsRNA molecules (of substantially greater than 21 to 23 base pairs in length) to identify a shorter DNA sequence more effective at dsRNA-mediated gene silencing. Other similar tools and improved or future tools created to assist in the selection of individual siRNAs can also be applied to longer (greater than 21 to 23 base pairs in length) dsRNAs. Thus, for example, it is possible to use averaged scores (e.g., average Reynolds scores) to select optimal sequences for gene silencing when using longer dsRNAs of greater than 21 to 23 base pairs in length. These results demonstrate that this method to provide improved DNA useful in dsRNA-mediated gene silencing may be applied to an initial DNA sequence including, surprisingly, substantially more than 21 to 23 nucleotides (such as, but not limited to, sequences of about 50 nucleotides, of about 100 nucleotides, and larger than at least between about 300 and about 330 nucleotides).

### Example 2

This example is a non-limiting example of a method to provide a DNA sequence for dsRNA-mediated gene silencing. More specifically, this example describes selection of an improved DNA useful in dsRNA-mediated gene silencing by (a) selecting from a target gene an initial DNA sequence including more than 21 nucleotides; (b) identifying at least one shorter DNA sequence derived from regions of the initial DNA sequence consisting of regions predicted to be more highly specific to said target gene; and (c) selecting a DNA sequence for dsRNA-mediated gene silencing that includes the at least one shorter DNA sequence.

A 2859 base pair sequence (SEQ ID NO. 18), which covered most of the coding region (3183 base pairs) of the target gene, maize (Zea mays) lysine ketoglutarate reductase/saccharopine dehydrogenase (LKR/SDH), was chosen as an initial DNA sequence. The LKR/SDH gene encodes a pre-protein for lysine ketoglutarate reductase (LKR) which is an enzyme capable of catabolizing lysine. Wild-type maize seed is relatively low in lysine, and it is of interest to modify maize seed lysine levels for use in animal feeds and other applications. Suppression of LKR and the resultant increase in lysine content is an example of an approach of modifying lysine content of maize seed.

Suppression of LKR can be effected, for example, by expressing in a plant a recombinant DNA construct containing a DNA sequence for dsRNA-mediated LKR silencing. The DNA sequence for dsRNA-mediated LKR silencing may be improved, for example, by designing the construct for increased specificity of maize LKR silencing. This may be accomplished in part by identifying regions of the initial DNA sequence that are predicted to be more highly specific to maize LKR, for example, regions substantially non-identical to non-target gene sequences.

In one non-limiting example, it may be desirable for the DNA sequence for dsRNA-mediated maize LKR silencing to include sequences substantially non-identical to gene sequences of plants other than maize (such as, but not limited to, other monocot plants including grasses and domestic grains other than maize, or dicot plants), or substantially non-identical to gene sequences of invertebrates (including, but not limited to, invertebrates that may feed on or otherwise come in contact with the transgenic maize, such as arthropods, annelids, nematodes, and molluscs). In another non-limiting example, where the transgenic maize seed is to be used as feed for domestic vertebrates (such as, but not limited to, fish, poultry, rabbits, swine, cattle, sheep, goats, horses, cats, and dogs), it may be desirable to minimize the potential to affect genes in the domestic vertebrates. In yet another non-limiting example, where the transgenic maize seed is expected to enter the human food supply, it may be desirable to minimize the potential to affect genes in humans.

One approach is to compare the proposed DNA sequence for dsRNA-mediated maize LKR silencing with known sequences of the non-target species (in the above examples, plants other than maize, invertebrates, domestic vertebrates, or humans), and to select maize LKR regions that are substantially non-identical to non-target gene sequences, for example, maize LKR regions within which every contiguous fragment including at least 19 nucleotides matches fewer than 19 (e.g., fewer than 19, fewer than 18, fewer than 17, or fewer than 16) out of 19 contiguous nucleotides of a non-target gene sequence. Another approach is to compare the proposed DNA sequence for dsRNA-mediated maize LKR silencing with known non-target gene sequences, and to select maize LKR regions within which every contiguous fragment including at least 21 nucleotides matches fewer than 21 (e.g., fewer than 21, fewer than 20, fewer than 19, fewer than 18, or fewer than 17) out of 21 contiguous nucleotides of a non-target gene sequence.

All possible contiguous 21-base pair segments of the 2859 base pair initial DNA sequence (SEQ ID NO. 18) were BLAST analyzed (Altschul et al. (1997) Nucleic Acids Res. 25:3389-3402, which is incorporated by reference herein) against a specificity sequence database that contained all expressed vertebrate gene sequences known at the time (represented as the expressed subset of the NCBI nr.nt collection (ncbi.nih.gov) and in-house proprietary cDNA and EST collections). The specificity database is updated on each running of the specificity tool and at the time of the analysis had 11.7 million sequences and 17.6 billion total bases.

The construct inserts matches were screened to find all perfect (21 out of 21) matches of 21 bases or longer, as well as their hit coordinates on the construct query sequences. Regions of these matches on the construct sequences were excluded as potential non-specific cross-match regions. The remaining regions were reported as “clear” for each query, that is to say, free of cross-match to the vertebrate sequences contained in the specificity database.

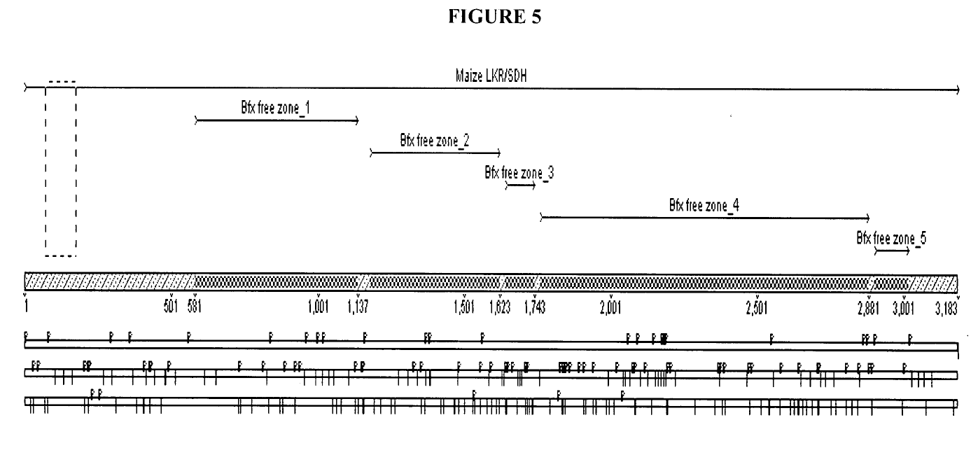

Five regions were identified as relatively large contiguous areas within which every contiguous fragment including at least 21 nucleotides matched fewer than 21 out of 21 contiguous nucleotides of known vertebrate gene sequences. These “bioinformatics-free” regions were designated “Bfx-free zone 1” (SEQ ID NO. 19), “Bfx-free zone 2” (SEQ ID NO. 20), “Bfx-free zone 3” (SEQ ID NO. 21), “Bfx-free zone 4” (SEQ ID NO. 22), “Bfx-free zone 5” (SEQ ID NO. 23), and are depicted graphically in FIG. 5. Additional smaller regions also containing 21-mer fragments “free” of a perfect (21 out of 21) match to known vertebrate sequences were also identified (data not shown). The five larger regions were chosen as preferred areas to use when designing an improved DNA useful in dsRNA-mediated gene silencing where it is desirable to minimize the potential to affect genes in vertebrates.

### Example 3

This example is a non-limiting example of a method to provide a DNA sequence for dsRNA-mediated gene silencing. More specifically, this example describes selection of an improved DNA useful in dsRNA-mediated gene silencing by (a) selecting from a target gene an initial DNA sequence including more than 21 nucleotides; (b) identifying at least one shorter DNA sequence derived from regions of the initial DNA sequence consisting of regions predicted to be more highly specific to the target gene; and (c) selecting a DNA sequence for dsRNA-mediated gene silencing that includes the at least one shorter DNA sequence.

In this non-limiting example, the partial cDNA sequence from a vacuolar ATPase gene (V-ATPase) from Western corn rootworm (WCR) (Diabrotica virgifera virgifera LeConte) was chosen as an initial DNA sequence (SEQ ID NO. 24). WCR V-ATPase has been demonstrated to function in a corn rootworm feeding assay to test dsRNA mediated silencing as a means of controlling larval growth.

This initial DNA sequence was screened for regions within which every contiguous fragment including at least 21 nucleotides matched fewer than 21 out of 21 contiguous nucleotides of known vertebrate sequences, and also screened for regions that did not contain sequence that encodes a potential polypeptide with perfect homology to a known allergen or toxin over 8 contiguous amino acids (or at least about 35% identity over at least about 80 amino acids). Three relatively large (greater than 100 base pair) regions that were free of such 21/21 hits were identified; these three shorter DNA sequences (SEQ ID NO. 25, SEQ ID NO. 26, and SEQ ID NO. 27) were combined by overlapped PCR to provide a novel chimeric DNA sequence (SEQ ID NO. 28) for dsRNA-mediated gene silencing.

The novel chimeric DNA sequence was tested in the same corn rootworm feeding assay. Samples of siRNA or full length double stranded RNA (dsRNA) were subjected to bioassay with a selected number of target pests. The controls were an untreated control (“UTC”, water) and an approximately 290 bp segment of the 3′ untranslated region (3′ UTR) of the vacuolar ATPase gene (V-ATPase) from Western corn rootworm. Varying does of dsRNA or siRNA were applied as an overlay to corn rootworm artificial diet according to the following procedure. Diabrotica virgifera virgifera (WCR) eggs were obtained from Crop Characteristics, Inc., Farmington, Minn. The non-diapausing WCR eggs were incubated in soil for about 13 days at 24 degrees Celsius, 60% relative humidity, in complete darkness. On day 13 the soil containing WCR eggs was placed between #30 and #60 mesh sieves and the eggs were washed out of the soil using a high pressure garden hose. The eggs were surface disinfested by soaking in Lysol for three minutes, rinsed three times with sterile water, washed one time with a 10% formalin solution and then rinsed three additional times in sterile water. Eggs treated in this way were dispensed onto sterile coffee filters and hatched overnight at 27 degrees Celsius, 60% relative humidity, in complete darkness.

Insect diet was prepared essentially according to Pleau et al. (2002) Entomologia Experimentalis et Applicata, 105:1-11, which is incorporated by reference herein, with the following modifications. 9.4 grams of Serva agar was dispensed into 540 milliliters of purified water and agitated until the agar was thoroughly distributed. The water/agar mixture was heated to boiling to completely dissolve the agar, then poured into a Waring blender. The blender was maintained at low speed while 62.7 grams of Bio-Serv diet mix (F9757), 3.75 grams lyophilized corn root, 1.25 milliliters of green food coloring, and 0.6 milliliters of formalin was added to the hot agar mixture. The mixture was then adjusted to pH 9.0 with the addition of a 10% potassium hydroxide stock solution. The approximately 600 milliliter volume of liquid diet was continually mixed at high speed and maintained at from about 48 degrees Celsius to about 60 degrees Celsius using a sterilized Nalgene-coated magnetic stir bar on a magnetic stirring hot plate while being dispensed in aliquots of 200 microliters into each well of Falcon 96-well round bottom microtiter plates. The diet in the plates was allowed to solidify and air dry in a sterile biohood for about ten minutes.

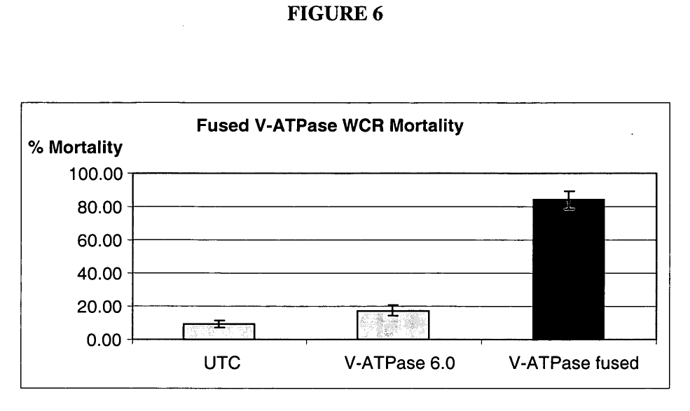

Thirty (30) microliter volumes of test samples containing either control reagents or double stranded RNA in varying quantities was overlaid onto the surface of the insect diet in each well using a micro-pipettor repeater. Insect diet was allowed to stand in a sterile biohood for up to one half hour after application of test samples to allow the reagents to diffuse into the diet and to allow the surface of the diet to dry. One WCR neonate larva was deposited to each well with a fine paintbrush. Plates were then sealed with Mylar and ventilated using an insect pin. 12-72 insect larvae were tested per dose depending on the design of the assay. The bioassay plates were incubated at 27 degrees Celsius, 60% relative humidity in complete darkness for 12-14 days. The number of surviving larvae per dose was recorded at the 12-14 day time point. Larval mass was determined using a suitable microbalance for each surviving larva. Data was analyzed using JMP©4 statistical software (SAS Institute, 1995) and a full factorial ANOVA was conducted with a Dunnet's test to look for treatment effects compared to the untreated control (p<0.05). A Tukey-Kramer post hoc test was performed to compare all pairs of the treatments (p<0.05). The results of this assay are shown in FIG. 6.

### Example 4

This example is a non-limiting example of a method to provide a DNA sequence for dsRNA-mediated gene silencing. More specifically, this example describes selection of an improved DNA useful in dsRNA-mediated gene silencing by (a) selecting from a target gene an initial DNA sequence including more than 21 nucleotides; (b) identifying at least one shorter DNA sequence derived from regions of the initial DNA sequence consisting of regions predicted to be more effective at dsRNA-mediated gene silencing; and (c) selecting a DNA sequence for dsRNA-mediated gene silencing that includes the at least one shorter DNA sequence.

Experiments similar to that described in Example 1 were carried out, again with the coding region of the target gene, firefly luciferase (luc) (SEQ ID NO. 1), was chosen as an initial DNA sequence including more than 21 nucleotides; in these experiments, oligonucleotides including 21-mers, 25-mers, or 27-mers were tested in transient assays using the protoplast system described in Example 1. The 25-mers were 21 base pair (bp) dsRNA with 2 bp overhangs at both the 5′ and 3′ ends; each 27-mer was a 25 bp sense strand that included 2 DNA nucleotides at the blunt 3′ end and a phosphate group on the 5′ end, and a 27 bp antisense strand. Use of a 2 bp overhang and DNA nucleotides at the blunt end has been reported to cause Dicer polarity, resulting in the production of a single 21 bp siRNA (see Rose et al. (2005) Nucleic Acids Res., 33:4140-4156). A 27-mer was found to suppress the target gene almost as effectively as an inverted repeat targeting luciferase, i.e., more effectively than corresponding 21-mers or 25-mers. This is in agreement with reports of improved silencing efficiency of 27-mers in animal systems (see Kim et al. (2005) Nature Biotechnol., 23:222-226). This experiment was repeated, with similar results observed.

Reynolds scores (see Reynolds et al. (2004) Nature Biotechnol., 22:326-330) were used to predict which 27-mer will be most effective at silencing luciferase in corn protoplasts. Seven 27-mers were tested: three 27-mers with high Reynolds scores, three 27-mers with low Reynolds scores and one 27-mer designed against GUS as a negative suppression control. In this experiment, the observed silencing efficacy correlated well with the efficacy predicted by Reynolds scores. The three 27-mers with the high Reynolds scores silenced efficiently (at the same level as the inverted repeat controls). The 27-mers with low Reynolds scores were not efficacious for silencing; they were comparable to the GUS 27-mer negative control.

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations may be applied to the materials and methods described herein without departing from the concept, spirit and scope of the invention. All such similar substitutes and modifications apparent to those skilled in the art are deemed to be within the spirit, scope and concept of the invention as defined by the appended claims.

What is claimed is:

1. A method to provide a DNA sequence for dsRNA-mediated gene silencing, comprising:
(a) selecting from a target gene an initial DNA sequence comprising more than 21 nucleotides;
(b) identifying at least one shorter DNA sequence derived from regions of said initial DNA sequence consisting of:
(i) regions predicted to be more effective at dsRNA-mediated gene silencing;
(ii) regions predicted to be more highly specific to said target gene; and
(iii) regions predicted to not generate undesirable polypeptides; and
(c) selecting a DNA sequence for dsRNA-mediated gene silencing that comprises said at least one shorter DNA sequence.

2. The method of claim 1, wherein said target gene is at least one eukaryote gene.

3. The method of claim 1, wherein said target gene is at least one non-eukaryote gene.

4. The method of claim 1, wherein said at least one shorter DNA sequence comprises at least 19 nucleotides.

5. The method of claim 1, wherein said at least one shorter DNA sequence comprises at least 21 nucleotides.

6. The method of claim 1, wherein said regions predicted to be more effective at dsRNA-mediated gene silencing comprise regions predicted to have higher siRNA efficiency.

7. The method of claim 1, wherein said regions predicted to be more highly specific to said target gene comprise regions substantially non-identical to a non-target gene sequence.

8. The method of claim 7 wherein said non-target gene sequence comprises a sequence of a species other than the species to which said target gene is endogenous.

9. The method of claim 7, wherein said regions substantially non-identical to a non-target gene sequence comprise regions within which every contiguous fragment comprising at least 19 nucleotides matches fewer than 19 out of 19 contiguous nucleotides of a non-target gene sequence.

10. The method of claim 7, wherein said regions substantially non-identical to a non-target gene sequence comprise regions within which every contiguous fragment comprising at least 21 nucleotides matches fewer than 21 out of 21 contiguous nucleotides of a non-target gene sequence.

11. The method of claim 1, wherein said undesirable polypeptides comprise polypeptides homologous to known allergenic polypeptides.

12. The method of claim 1, wherein said undesirable polypeptides comprise polypeptides homologous to known polypeptide toxins.

13. The method of claim 1, wherein said DNA sequence for dsRNA-mediated gene silencing comprises a chimeric sequence.

14. The method of claim 13, wherein said chimeric sequence comprises more than one shorter DNA sequence.

15. The method of claim 1, wherein said target gene comprises multiple target genes.

16. A transgenic eukaryote whose genome comprises at least one DNA sequence for dsRNA-mediated gene silencing provided by the method of claim 1.

17. The transgenic eukaryote of 16, wherein said transgenic eukaryote comprises a plant.

18. The transgenic eukaryote of 16, wherein said at least one target gene is at least one eukaryote gene.

19. The transgenic eukaryote of 16, wherein said at least one target gene is at least one non-eukaryote gene.
